(19) United States
(12) Patent Application Publication
(10) Pub. No.: US 2005/0261719 A1
(43) Pub. Date: Nov. 24, 2005

(54) Catheter and method of its use

(76) Inventor: Israel Chermoni, Haifa (IL)

Correspondence Address: BROWDY AND NEIMARK, P.L.L.C., 624 Ninth Street, N.W., Washington, DC, 20001, US

(21) Appl. No.: 10/303,064

(22) Filed: Nov. 25, 2002

(51) Int. Cl. A61M 29/00

(52) U.S. Cl. 606/191

(57) ABSTRACT

A catheter capable of being propelled through blood vessels. The catheter comprises at its distal end a head portion and, proximal thereto, a propulsion compartment. The propulsion compartment comprises an outer tube and an inner tube, the tubes being concentric, wherein one of the inner or outer tubes can slidingly move in relation to the other of the inner or outer tubes in response to a pressure exerted thereon by a fluid introduced into one or both of the tubes. A method for propelling a catheter through the lumen of a body vessel is also disclosed.

### FIELD OF THE INVENTION

This invention relates to a method and apparatus for use in catheterization.

### BACKGROUND OF THE INVENTION

Cardiac catheterization is a frequently used technique by which a catheter is inserted into an artery or vein up to and beyond the heart. It may be used for various procedures such as balloon angioplasty (PTCA), placing a stent in a blood vessel, injecting drugs into the heart, measuring cardiac blood flow and blood pressure, cardioplegia, various diagnostic procedures and inserting an electrode into the heart.

In this procedure, the catheter is advanced through the vascular system by pushing the end of the catheter tube from outside the body. A major difficulty in carrying out the catheterization procedure is in maneuvering the catheter through the tortuous and narrow blood vessels. As is well known, blood vessels are quite serpentine and winding, often having sharp turns therein. In addition, many blood vessels are tapered and conventional catheters have difficulty in moving through blood vessels having a diameter of less than 2.5 mm. Approximately 20% of catheterization procedures involve such tortuous and narrow blood vessels.

U.S. Pat. No. 4,762,129 to Bonzel discloses a dilatation catheter which includes a balloon capable of being enlarged by injecting a fluid through an aspiration tube that terminates in the balloon. The aspiration tube, which is reinforced with a stabilizing wire, also serves to transmit thrust and tension forces to push and pull the balloon to and fro and rotate it on the guide wire.

U.S. Pat. No. 4,998,916 to Hammerslag and Hanmmerslag discloses an elongate steerable implement such as a catheter for coronary angioplasty applications. A floppy steerable tip on a steering region at the distal end of the implement and a control device at the proximal end are connected by means of a plurality of axially movable deflection wires extending throughout the implement.

U.S. Pat. No. 5,324,260 to O'Neill et al, discloses a coronary simus catheter for the retrograde infusion of cardioplegia solutions into the coronary sinus. The catheter comprises a catheter tube having infusion, pressure sensing and balloon inflation lumens and a pressure sensor tube in fluid communication with the balloon-inflation lumen for sensing pressure in the inflatable balloon.

U.S. Pat. No. 5,439,445 to Kontos discloses a support catheter assembly for facilitating medical procedures. The assembly includes a tubular body and a manipulating member connected thereto for inserting, advancing, withdrawing and maneuvering the body during the procedure. The manipulating member may be a wire or a manipulating tube.

EP 620,016 to Weber discloses a hydrodynamic suction catheter for the removal of blood clots and thrombi which have been dislodged from the blood vessel wall. A liquid jet pump creates a vacuum due to the flow of liquid through a pressure channel in the distal tip of the catheter.

### SUMMARY OF THE INVENTION

It is an object of the present invention to provide a catheter of reduced diameter.

It is a further object of the invention to provide a catheter capable of negotiating curved blood vessels.

It is a still fiber object of the invention to provide a method for propelling a catheter through a blood vessel.

In a first aspect of the invention, there is provided a catheter comprising at its distal end a head portion and, proximal thereto, a propulsion compartment. The propulsion compartment comprises an outer tube and an inner tube, the tubes being concentric, wherein one of the inner or outer tubes can slidingly move in relation to the other of the inner or outer tubes in response to a pressure exerted thereon by a fluid introduced into one or both of the tubes.

The present invention relates to an apparatus for use in catherization techniques in general, and cardiac catherization in particular. The catheter of the invention differs from conventional catheters in that the catheter is pulled through the blood vessels from the anterior end rather than being pushed from the posterior end. The difference is comparable to the advantage of using front wheel drive in a vehicle as compared to conventional rear wheel drive. Thus, only the head portion must be pushed through an obstacle such as a sharp curve, while the remaining tubing is pulled thereafter.

Due to the method of propulsion of the catheter of the invention by pulling rather than by pushing, the catheter may be less rigid and more flexible than the conventional catheter. Thus, the catheter may have a reduced diameter which enables it to enter very small blood vessels such as arterioles. In a preferred embodiment the catheter has a diameter of less than 1.5 mm.

The head portion of the catheter may fulfill any function required of the catheter. Examples of head portions which may be used in the invention include an inflatable balloon, a stent carrier, a drug delivery device, a measuring device, an electrical device and an optical device. The catheter of the invention is not limited to a specific type of head portion.

The propulsion compartment comprises two concentric tubes. These tubes are preferably manufactured from a flexible but firm material such as flexible plastic or rubber. Usually, one of the tubes is sealed at its distal end. In a preferred embodiment, the tube which is sealed at its distal end is the tube which moves forward relative to the other tube. In other words, the sealed tube propels the head portion forward into the body vessel lumen while the unsealed tube generally remains outside the body. The proximal ends of the tubes always remain outside the body.

The catheter may also comprise a coaxial delivery tube connected to the head portion. The delivery tube functions to allow communication with the head portion when it is inside the body. For example, in the case where the head portion is a balloon, the delivery tube acts as a feed tube to feed fluid to and from the balloon to inflate and deflate it, as needed. In the case where the head portion is an optical device, the optical fibers or wires extend through the delivery tube. Other possibilities will be clear to the skilled man of the art.

The fluid introduced into the tubes may be any fluid compatible with the catheter material. Although the structure of the catheter prevents leakage of the fluid into the body lumen, in a preferred embodiment the fluid is saline, most preferably sterile saline. The fluid may be provided by any fluid compressing apparatus such as a fluid reservoir or pump which may be manual or automatic. A non-limiting example of a fluid reservoir is a syringe.

In a second aspect of the invention, there is provided a method for propelling a catheter through the lumen of a body vessel comprising: (a) inserting a guide wire into the lumen; (b) mounting a catheter according to the invention on the guide wire, and (c) injecting a fluid into the propulsion compartment of the catheter, thereby propelling the catheter through the lumen of the body vessel.

### BRIEF DESCRIPTION OF THE DRAWINGS

In order to understand the invention and to see how it may be carried out in practice, preferred embodiments will now be described, by way of non-limiting example only, with reference to the accompanying drawings, in which:

FIG. 1 is a side sectional view of a catheter according to one embodiment of the invention;

FIG. 2 is an enlarged partial view of the catheter of FIG. 1;

FIGS. 3A & 3B illustrate the insertion of the catheter of FIG. 1 into a blood vessel. FIG. 3A shows a side sectional view of the catheter prior to insertion and FIG. 3B shows the catheter after insertion;

FIG. 4 is a side sectional view of a catheter according to another embodiment of the invention;

FIGS. 5A & 5B illustrate the insertion of the catheter of FIG. 4 into a blood vessel. FIG. 5A shows a side sectional view of the catheter prior to insertion and FIG. 5B shows the catheter after insertion;

FIGS. 6 and 7 are side sectional views illustrating how the balloon of the catheter of FIGS. 5 is inflated; and

FIG. 8 illustrates a further embodiment of a catheter according to the invention.

### DETAILED DESCRIPTION OF THE INVENTION

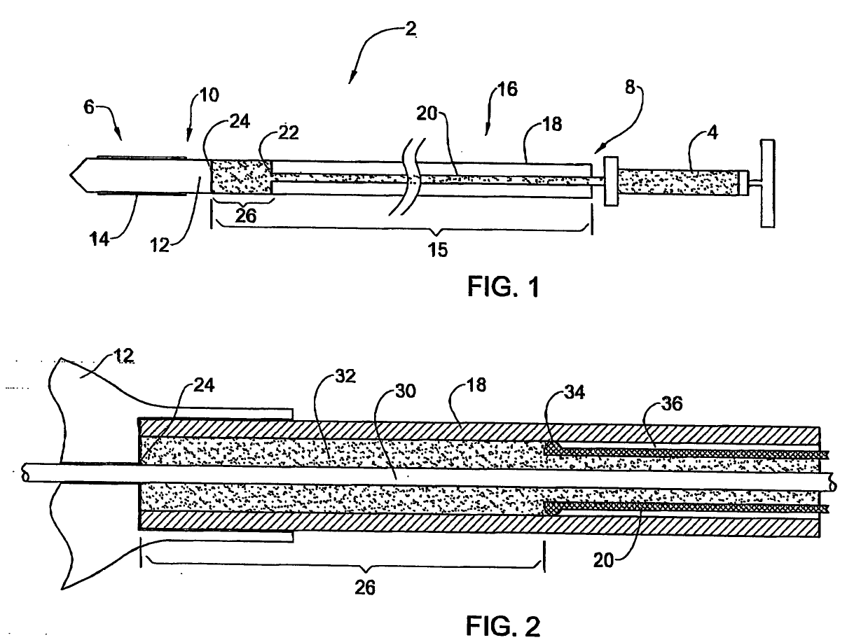

One embodiment of a catheter according to the invention is illustrated in FIG. 1, which shows the catheter 2 connected to a fluid reservoir 4 (shown in the figure as a syringe). In the present specification, the distal end of the catheter refers to the end which is first inserted into the body, while the proximal end is the end facing the person who inserts the catheter. Thus, the catheter shown in FIG. 1 has a distal end 6 and proximal 8 end. At the distal end 6 of the catheter 2 is a head portion 10, being in this example an inflatable balloon 12 on which is mounted a stent 14 for placement in an occluded artery. It will be clear to the skilled man of the art that the head portion may serve a variety of functions as is known in the art of catherization.

Proximal to the head portion is a propulsion compartment 15 comprising two concentric tubes: an outer tube 18 and an inner tube 20 having a smaller diameter and being located within the outer tube. The diameter of the outer tube will generally be less than 1.5 mm, preferably in the range of 1.2-1.5 mm. The diameter of the inner tube will generally be in the range of 0.9-1.3 mm. The catheter of this embodiment is suitable for use in blood vessels having a diameter of approximately 2.5 mm or more. The length of the tubes is at least the length which is to be traveled by the catheter in the body vessel (e.g. arteries). In a preferred embodiment, the length is in the range of 1-4 m. The tubes can slidingly move in relation to each other, as will be explained in more detail below. The distal end 22 of the inner tube 20 is open and proximally spaced from the distal end 24 of the outer tube 18, which is sealed. An interval 26 is formed within the outer tube between the distal ends of the inner and outer tubes. The fluid reservoir is in fluid communication with the proximal end of the inner tube 20 so that when the fluid reservoir is filled with fluid and pressure is applied thereby, the fluid flows through the inner tube 20 into the interval 26. The fluid, however, cannot pass beyond the sealed distal end 24 of the outer tube.

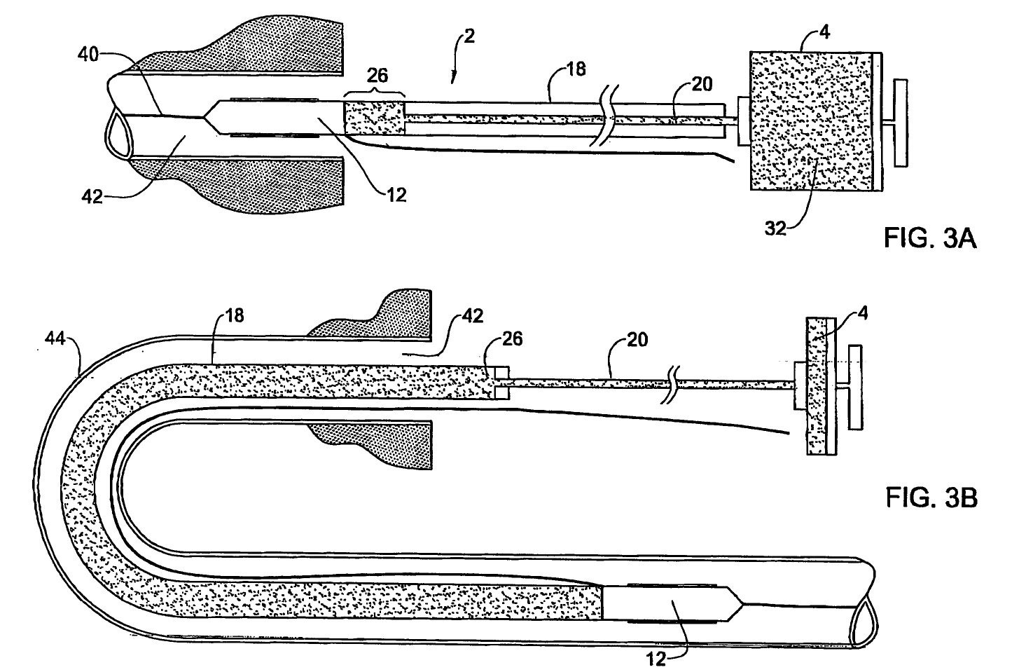

FIG. 2 shows a portion of the catheter in greater detail. As in FIG. 1, the catheter comprises an inflatable balloon 12 (only the proximal end is shown) attached at its proximal end to the outer tube 18, which contains within it the inner tube 20. Passing through and within the inner and outer tubes is a feed tube 30 which is in fluid communication with the interior of the balloon 12. The function of the feed tube is to convey a fluid into the balloon in order to inflate it at the site of a stenosis. The feed tube can serve other functions depending on the function of the head portion.

A fluid 32 may be pumped through the inner tube 20 into the interval 26. The distal end of the inner tube is surrounded by a sealing ring 34 which functions to prevent the fluid from returning along the space 36 between the inner and outer tubes. However, the sealing ring does not prevent movement of the outer and inner tubes in relation to each other. The fluid is also prevented from egressing through the distal end 24 of the outer tube, which is sealed. Thus, infusion of the fluid 32 into the interval 26 results in an increase in fluid pressure in the interval 26, which is relieved by the forward movement of the outer tube 18 relative to the inner tube 20.

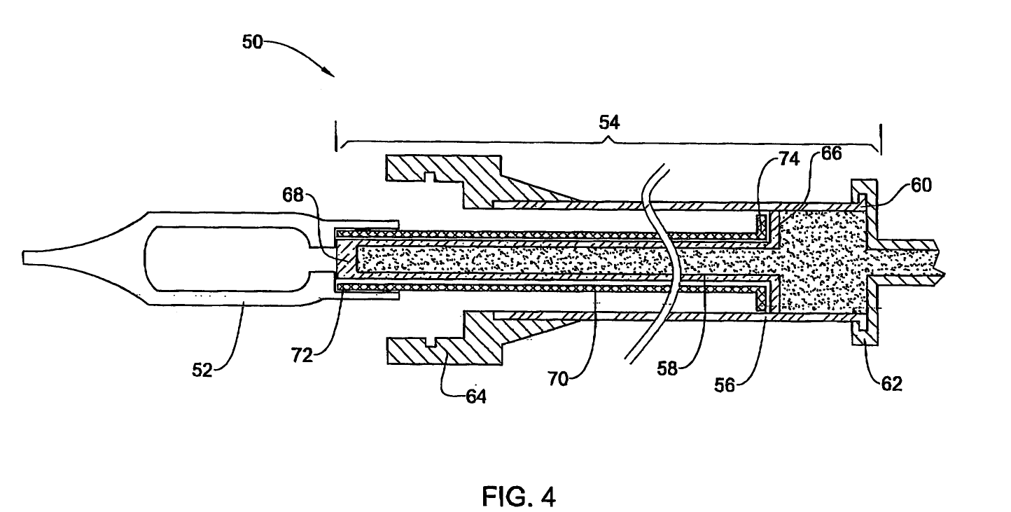

The operation of the catheter of FIGS. 1 & 2 is illustrated in FIGS. 3A and 3B. Referring first to FIG. 3A, a guide wire 40 is inserted into a body lumen 42 such as of an appropriate artery or vein (e.g. percutaneously or via a body orifice), usually through a guide catheter (not shown), as is well known in the art. The catheter 2 is mounted on the guide wire and positioned within the entrance to the lumen. As stated above, the length of the outer 18 and inner 20 tubes is at least the length to be traveled in the lumen. The fluid reservoir 4 is in a filled state and the fluid 32 has entered the inner tube 20 and interval 26.

FIG. 3B shows the situation after most of the contents of the reservoir has been transferred into the catheter. The creation of fluid pressure in the interval 26 distal to the inner tube 20 urges the outer tube 18 and balloon 12 forward, thereby increasing the volume of the interval 26 and relieving the pressure. The continuous introduction of fluid into the inner tube causes the balloon to advance until it reaches the desired location in the blood vessel. The advancing balloon pulls the remainder of the catheter behind it so that it passes through sharp curves 44 in the blood vessel with greater ease than if the propulsion was by pushing the catheter from behind. Once the catheter reaches the desired location in the blood vessel, the balloon 12 may be inflated through the feed tube. At completion of the medical procedure, the catheter is manually removed from the body by simply pulling it out.

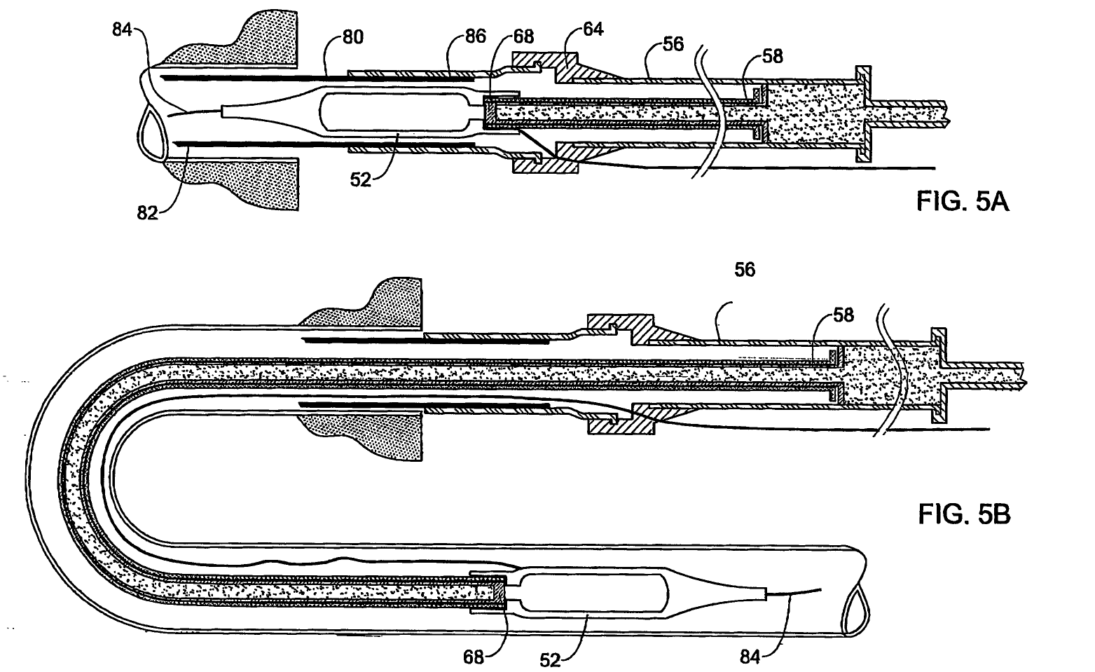

A second embodiment of a catheter according to the invention is illustrated in In FIG. 4. As in the first embodiment, the catheter 50 comprises a head portion (being an inflatable balloon 52) at the distal end and a propulsion compartment 54 proximal thereto. The propulsion compartment comprises an outer tube 56 and an inner tube 58, the two tubes being concentric. The proximal end of the outer tube 56 has a flange 60 to which may be attached an adapter 62 for connecting the catheter to a fluid reservoir (not shown). The distal end 64 of the outer tube is adapted to be attached to the proximal end of a guide catheter, as will be discussed below.

The proximal end of the inner tube ends in a sealing ring 66 sealing the space between the outer and inner tubes, but allowing relative movement between them. The distal end 68 of the inner tube is sealed. Fluid from the reservoir can flow into the outer tube 56 and from there into the inner tube 58. A preferred diameter of the catheter in this embodiment is 0.6-1.0 mm. The catheter of this embodiment is suitable for use in blood vessels having a diameter of approximately 1.5 mm or more. In this embodiment, there is no additional feed tube leading to the head portion, thus enabling the diameter of the catheter to be reduced. There is, however, an open ended delivery tube 70 which is connected at its distal end 72 to the head portion. The inner tube is inserted within the delivery tube, and is freely movable therein. The proximal end 74 of the delivery tube ends in a flange distal to the proximal end 66 of the inner tube. The length of the inner tube 58 and delivery tube 70 is at least the length which is to be traveled by the catheter in the body vessel.

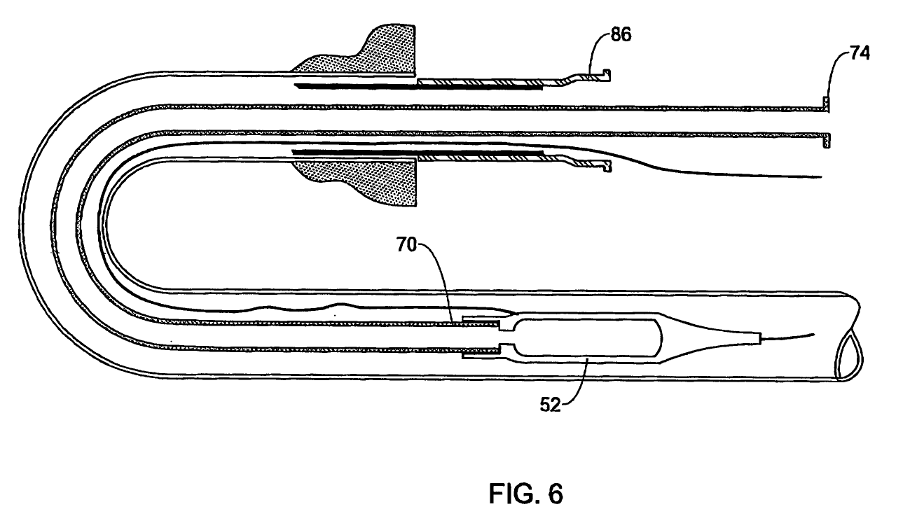

The operation of the catheter according to this embodiment is illustrated in FIGS. 5A and 5B. In FIG. 5A, a guide catheter 80 has been inserted into the body lumen 82 and a guide wire 84 has been inserted through the guide catheter into the lumen. The distal end 64 of the outer tube 56 of the catheter is attached to an adapter 86 at the proximal end of the guide catheter 80. The head portion (balloon 52) of the catheter is located at the entrance to the body vessel.

Fluid is then pumped from the fluid reservoir into the outer tube 56, flowing from there into the inner tube 58. Since the distal end 68 of the inner tube is sealed, there is a build up of pressure in the propulsion compartment which is relieved by the forward movement of the inner tube, as illustrated in FIG. 5B. The inner tube pushes the balloon 52 forward along the guide wire, and the balloon pulls the remainder of the catheter after it.

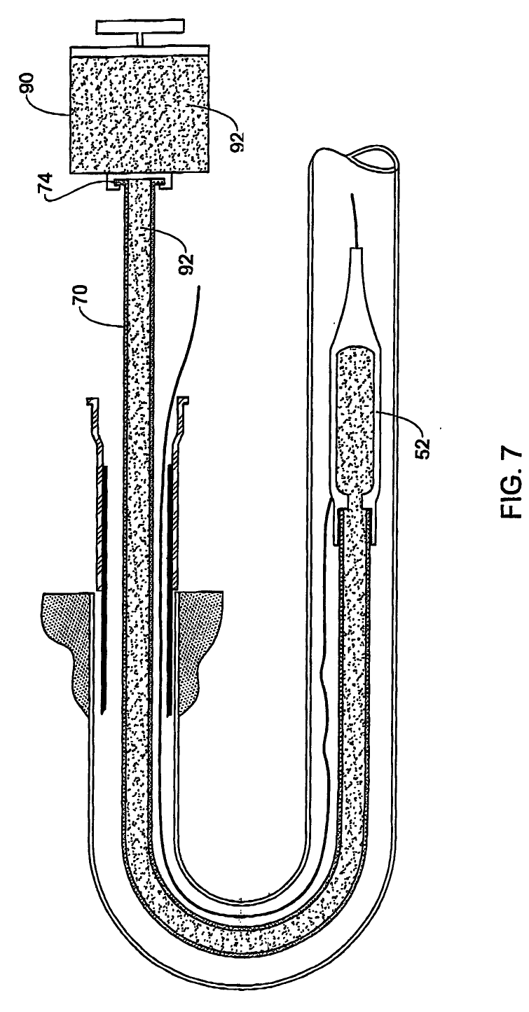

Once the catheter is at the required site in the body vessel (i.e. a stenosis in an artery), it may be necessary to inflate the balloon at the head of the catheter (or carry out some other function depending on the identity of the head). In order to inflate the balloon, the outer tube is detached from the guide catheter adapter end 86, and the inner tube and outer tube are removed, as shown in FIG. 6. Thus, what remains in the body vessel lumen are the balloon 52 with the delivery tube 70 attached thereto. A fluid reservoir 90 is then attached to the flanged end 74 of the delivery tube 70, as shown in FIG. 7, and fluid 92 in the reservoir can be introduced into the balloon 52 through the delivery tube 70, thereby inflating the balloon. As will be understood by the skilled man of the art, other types of catheter heads may be used, and necessary communication with the head may be carried out through the delivery tube.

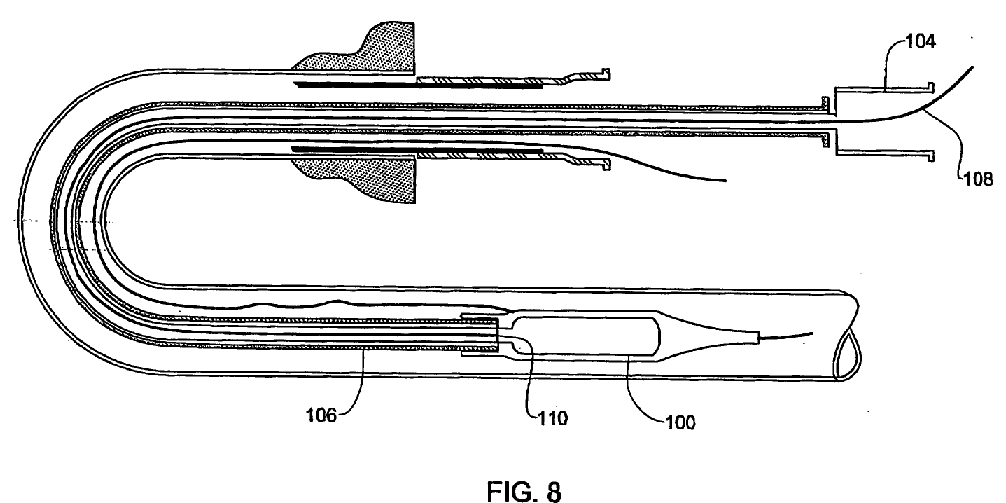

A further embodiment of the invention is illustrated in FIG. 8. This embodiment is to a catheter comprising a head compartment 100, and a propulsion compartment which consists of an inner tube 104 having a sealed distal end 110 and a delivery tube 106, as described previously with respect to the embodiment of FIG. 4. In this embodiment, a thin, extended, flexible but firm object, such as a cable 108, is inserted into the inner tube up to its distal end 110 and is used instead of fluid pressure to advance the head compartment by pushing it. The head compartment then pulls after it the remainder of the catheter.

1. A catheter comprising at its distal end a head portion and, proximal thereto, a propulsion compartment, said propulsion compartment comprising an outer tube and an inner tube, said tubes being concentric, wherein one of said inner or outer tubes can slidingly move in relation to the other of said inner or outer tubes in response to a pressure exerted thereon by a fluid introduced into one or both of said tubes.

2. The catheter according to claim I wherein said head portion is selected from the group consisting of an inflatable balloon, a stent carrier, a drug delivery device, a measuring device, an electrical device and an optical device.

3. The catheter according to claim 1 wherein one or both of said inner and outer tubes contains therewithin a coaxial delivery tube connected to said head portion.

4. The catheter according to claim 1 wherein one of said inner or outer tubes is sealed at its distal end.

5. The catheter according to claim 4 wherein the tube which is sealed at its distal end is the tube which moves relative to the other tube.

6. The catheter according to claim 1 having a diameter less than 1.5 mm.

7. The catheter according to claim 1 manufactured using a flexible but firm material.

8. The catheter according to claim 7 wherein said material is selected from the group consisting of flexible plastic and rubber.

9. A catheter comprising at its distal end a head portion and, proximal thereto, a propulsion compartment, said propulsion compartment comprising a tube, said tube being sealed at its distal end, and a flexible cable capable of being inserted into said tube.

10. A method for propelling a catheter through the lumen of a body vessel comprising:
(a) inserting a guide wire into the lumen;
(b) mounting a catheter on said guide wire, wherein said catheter comprises at its distal end a head portion and, proximal thereto, a propulsion compartment, said propulsion compartment comprising an outer tube and an inner tube, said tubes being concentric, wherein one of said inner or outer tubes can slidingly move in relation to the other of said inner or outer tubes in response to a pressure exerted thereon by a fluid introduced into one or both of said tubes; and
(c) injecting a fluid into the propulsion compartment of said catheter, thereby propelling the catheter through the lumen of the body vessel.
